(19) United States
(12) Patent Application Publication
(10) Pub. No.: US 2014/0275974 A1
(43) Pub. Date: Sep. 18, 2014

(54) Surgical Navigation Systems and Methods

(71) Applicant: Mark Alan Samuels, Johns Creek, GA (US)

(72) Inventor: Mark Alan Samuels, Johns Creek, GA (US)

(21) Appl. No.: 14/206,419

(22) Filed: Mar. 12, 2014

Related U.S. Application Data
(60) Provisional application No. 61/852,375, filed on Mar. 15, 2013.

(51) Int. Cl. A61B 5/107; A61B 19/00; A61M 31/00; A61B 5/055; A61B 6/03; A61B 5/00; A61B 8/08

(52) U.S. Cl. CPC A61B 5/1076; A61B 5/6853; A61B 19/54; A61B 8/0841; A61B 5/055; A61B 6/03; A61B 5/0077; A61B 5/0073; A61M 31/00; A61B 19/5244; A61B 2019/5466; A61B 2019/5454; A61B 2019/5425; A61B 2019/5437; A61B 2019/5412; A61B 2019/5483; A61B 2019/5265
USPC 600/417; 600/587; 600/424

(57) ABSTRACT

Implementations described herein relate to an anatomical referencing system and associated methods for surgical navigation that include referencing shapes and features on excised target tissue back to the wound or cavity in the body from which it was removed. Such an anatomical referencing system can comprise an expandable structure operably coupled to a controller assembly. The expandable structure can have a port in communication with an interior cavity and a surface adapted for measuring having a plurality of navigation markers. The controller assembly can operate to communicate with the expandable structure through the port and can have a means for expanding the expandable structure to a selected pressure against the cavity. The expandable structure can be adapted to conform to the geometry of the cavity in which it is placed upon expansion by the controller assembly. The controller assembly can include a means for recording the dimensions of the interior cavity of the expanded structure.

### CROSS-REFERENCE TO RELATED APPLICATION

This application claims priority to U.S. Provisional Patent 61/852,375 entitled “Margin Navigation System” and filed on Mar. 15, 2013, which is herein incorporated by reference in its entirety.

### BACKGROUND

1. The Field of the Invention

Implementations described herein relate generally to an anatomical referencing system and associated methods for surgical navigation that include referencing shapes and features on excised tissue back to the wound or cavity in the body from which it was removed.

2. Related Art

Using conventional techniques for lumpectomy and other surgical procedures, a reoperation to remove missed cancers is needed in up to 50% of the cases. Information on exactly where the remaining cancer is located in the wound cavity after tumor removal is very general and not specific. There are many methods that are currently employed for marking excised tissue as to its orientation with regard to the body so that features on the excised tissue (such as the presence of cancer) can be referenced back to features remaining on the body. Such information is useful for identifying the location of additional tissue to be removed in the case that cancer is found on the outside margin of an excised tumor. Excised tissue is generally thought of as having six sides like a cube and in the case of lumpectomy are referenced as follows: the anterior, posterior, superior, inferior, medial, and lateral. Techniques that are currently employed include, for example, selectively placing a unique suture stitch on two or more sides of the excised tissue, selectively placing clips or other metal markers on two or more sides and selectively placing colored ink on the excised tissue. Unfortunately, excised tissue samples are irregularly shaped and the exact demarcation between the sides is ambiguous which can lead to a faulty result. For example, a tumor that is located on the dividing line between the superior and the posterior surface may be misidentified leading a surgeon to remove healthy tissue instead of cancerous tissue during a reoperation.

A need therefore exists for systems and methods for creating a three-dimensional map or representation of the excised tissue or material and the cavity from which it was removed.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Implementations of the present disclosure include an anatomical referencing system comprising an expandable structure operably coupled to a controller assembly. In some aspects, the expandable structure can have a port in communication with an interior cavity and a surface adapted for measuring having a plurality of navigation markers. In other aspects, the controller assembly can operate to communicate with the expandable structure through the port and can have a means for expanding the expandable structure to a selected pressure against the cavity. In a further aspect, the controller assembly can include a means for measuring at least one of the topology and the three-dimensional geometry of the surface of the expandable structure. It is contemplated that the expandable structure be adapted to conform to the geometry of the cavity in which it is placed upon expansion by the controller assembly. It is also contemplated that the means for measuring at least one of the topology and the three-dimensional geometry of the surface of the expandable structure can be adapted to record the dimensions of the interior cavity of the expanded structure.

In other aspects, the present disclosure provides a method for surgical navigation comprising the steps of excising a target tissue from a patient, measuring the dimensional characteristics of the target tissue, examining the tissue to locate any positive margins, using the dimensional characteristics of the target tissue and the locations of any positive margins thereon to construct a three-dimensional map of the excised tissue to guide a surgery. In further aspects, an anatomical referencing system according to the present disclosure can be provided. Here, the expandable structure can be inserted into the cavity, the cavity closed, the controller assembly caused to expand the expandable structure to a selected pressure and record the dimensions of the interior cavity of the expandable structure and the locations of each of the plurality of navigational markers. Then, a three-dimensional map of the interior cavity of the expandable structure optimally and the locations of each of the plurality of navigational markers. The resulting three-dimensional map of the target excised tissue and the three-dimensional map of the cavity can be correlated with each other to reference positive margins on the excised tissue back to the corresponding cavity margins to create a surgical model; and the surgical model used to guide a surgery.

In certain aspects, the present disclosure relates to a system for referencing the precise location of cancer found in or on the margins of an excised tissue sample back to the location of the remaining cancer in the body.

In other aspects, the present disclosure provides systems and methods to reference the location of a positive margin back to the exact location in the wound cavity where it occurred. In further aspects, the present disclosure provides for systems and methods to at least one of improve cosmetic results, to form custom implants, to improve survival rates and to reduce scar formation subsequent to an operation.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates an anatomical referencing system comprising an expandable structure conformed to a wound cavity of a human or animal, where the wound cavity is the result of target tissue removal, having a plurality of navigational markers on the expandable structure and a controller assembly having a laser profiling device inserted into the expandable structure for recording the dimensions of the interior cavity of the expandable structure and the locations of each of the plurality of navigational markers. The controller assembly is further adapted to analyze the data from the laser profiling device and display a three-dimensional model constructed from the data thereof.

FIG. 2 shows the expandable structure having a plurality of navigational marks conforming to the wound cavity profile.

FIG. 3 shows the removed target tissue having a positive margin identified for reference back to the navigational markers of the three-dimensional model of the wound cavity and a laser profiling device for developing a 3-D model of the removed target tissue.

### DESCRIPTION OF THE INVENTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well-known aspects of spectroscopic instrumentation and calibration thereof have not been described in particular detail in order to avoid unnecessarily obscuring aspects of the disclosed implementations.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

As used in the specification and the appended claims, the term “lumpectomy” should be construed as any surgical procedure designed to remove a target tissue from an affected woman's or man's breast. The target tissue can comprise a tumor, cancerous cells or suspected cancerous cells.

As used in the specification and the appended claims, the terms “wound”, “cavity” and “wound cavity” are used interchangeably and should be construed to refer to the hole or void that is left after, e.g., a target tissue is removed from the body.

As used in the specification and the appended claims, the phrases “anatomic referencing” and “anatomically referenced” should be construed to refer to the process of correlating a feature on a target tissue back to a corresponding location in the wound cavity.

As used in the specification and the appended claims, the term “expandable structure” should be construed to refer to a flexible structure that can be expanded or inflated and can comprise, for example and without limitation, a balloon, a mesh bag and the like.

As used in the specification and the appended claims, the term “3-D scanner” should be construed to refer to a device that employs any number of techniques to measure the inner or outer dimensions of an object in three dimensions and can comprise equipment employing, for example and without limitation, ultrasound, lasers, triangulation, a charge-coupled device, x-ray, CAT, MRI, cameras and the like.

As used in the specification and the appended claims, the term “digital pathology software” should be construed to refer to a system designed to evaluate a pathology image generated from a tissue sample.

As used in the specification and the appended claims, the term “radio-opaque” should be construed to refer to the ability of an object to obstruct the passage of radiant energy, e.g., x-rays, and the obstructed areas being recorded distinctly on a resultant image.

As used in the specification and the appended claims, the term “positive margin” or “dirty margin” should be construed to refer to an area of target tissue in which abnormal or cancerous tissue is present on or near the outside, wherein the presence of abnormal or cancerous tissue can indicate that additional cancer or abnormal tissue may still be left in the body.

As used in the specification and the appended claims, the term “brachytherapy” should be construed to refer to a technique of delivering radiation therapy directly to the site of a target tissue.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be predefined it is understood that each of these additional steps can be predefined with any specific aspect or combination of aspects of the disclosed methods.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be predefined it is understood that each of these additional steps can be predefined with any specific aspect or combination of aspects of the disclosed methods.

Reference will now be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well known aspects of spectroscopic instrumentation and calibration thereof have not been described in particular detail in order to avoid unnecessarily obscuring aspects of the disclosed implementations.

Turning now to FIG. 1, one implementation of the present disclosure comprises an anatomical referencing system 100 comprising an expandable structure 102 and a controller assembly 104. In one aspect, the expandable structure 102 has a port 106 in communication with an interior cavity 108 and a surface adapted for measuring having a plurality of navigational markers 110. In another aspect, the controller assembly 104 is adapted to communicate with the expandable structure through the port and comprises a means for expanding the expandable structure to a selected pressure against the wound cavity 112. In a further aspect, the controller assembly comprises a means for recording the dimensions of the interior cavity 108 of the expandable structure. It is contemplated that the expandable structure is adapted to conform to the geometry of the wound cavity 112 upon expansion. It is further contemplated that the means for measuring at least one of the topology and the three-dimensional geometry of the surface of the expandable structure is adapted to record the dimensions of the interior cavity 108 of the expanded structure.

In operation, a target tissue is removed and the expandable structure 102 is placed in the resultant wound cavity 112. In light of the present disclosure, one skilled in the art will appreciate that the wound cavity will be accessible through a skin access flap and that the wound cavity has an arbitrary (and at this point unknown) relationship to both the excised target tissue and the body. The skin access flap can be sutured closed to provide for substantial confinement of the expandable structure 102 within the cavity 112. Next, the expandable structure 102 can be expanded to a preselected pressure such that it conforms to the wound cavity 112. Once the controller assembly has expanded the expandable structure to the correct pressure and the expandable structure conforms to the interior cavity 108, a means for recording the dimensions of the interior cavity is inserted into the expandable structure through the port and the dimensions recorded. The controller assembly can then use the recorded dimensions to construct a 3-D map of the interior cavity showing the locations of the plurality of navigational markers 110. It is further contemplated that the controller assembly can further comprise a means for displaying the 3-D map 112.

One skilled in the art will appreciate that current techniques to reference pathology results back to the point of excision are very poor today. This can be due to the fact that the target tissue (or tumor) cavity from which the target tissue is removed is surrounded by soft tissue, leading the cavity to deform once the tumor tissue is removed. Such deformation interferes with the anatomic referencing. In addition, the target tissue tends to deform once it is removed from the body making it difficult to determine exactly how it references back to the removal site. To avoid this deformation in the wound cavity, a positive pressure is applied to the inside of the cavity where the positive pressure is selected to match the tissue characteristics. Acta Orthop. Scan. 57, 444-446, 1986 describes internal tumor pressures and is hereby incorporated by reference in its entirety. Accordingly, it is contemplated that the preselected pressure can be from about 0.1 to about 70 mmHg, and, more preferably from about 2 to about 5 mmHg. It is also contemplated that the expandable structure be inflated to a pressure not greater than the interstitial pressure of the target tissue (prior to removal) by the action of the controller assembly 104.

In aspects of the present invention, the means for recording the dimensions of the interior cavity of the expandable structure can be, for example and without limitation, a three-dimensional scanner, a camera, an ultrasound measuring device, an MRI device, a CAT scan device, an x-ray device and the like. One skilled in the art will also appreciate that any of the disclosed means can measure the dimensional characteristics of an irregularly-shaped object such as the excised target tissue. In one exemplary aspect, a laser or ccd device can employ a simple triangulation system to measure distance from N points on a surface of the expandable structure and those N points connected or interpolated to form a 3-D or topographical representation of the expandable structure 102 and, thus, the cavity 112 to which it conforms.

In a further aspect, the means for recording the dimensions of the interior cavity can be further adapted to record the locations of each of the plurality of navigational markers.

In aspects of the present invention, a 3-D scanner can be positioned in the expanded expandable structure that is coated with a reflective material configured to facilitate the measurement of the expandable structure to create a 3-D data map of the shape of the interior of the cavity. One illustrative examples of this can be, for example and without limitation, Lantos Technologies' AURA™ 3-D Ear Scanning System and devices sold by 3-DM Systems (formerly ShapeStart Measurement Systems).

In aspects of the present invention, the means for inflating the expandable structure can be, for example and without limitation, a pressure pump, a syringe, selectively-controlled temperature changes, a selectively-controlled chemical reaction, a selectively-controlled mechanical force and the like.

FIG. 2 shows additional detail of the expandable structure 102 and the navigational markers 110 that can be employed to reference the location of any positive margins back the point in the body from which it came. The navigational markers are designed to be visible on, for example and without limitation, an x-ray, a CT scan, an MRI, a 3-D scanner and the like. In light of the present disclosure, one skilled in the art will appreciate that the shape and position of the conformed expandable structure enables any of the navigational markers to be referenced to the excised target tissue or 3-D representations thereof, allowing correlation of positive margins back to the wound cavity. In one further and illustrative example, a surgical marker can be placed at the position of the navigation marker identified as the number 2. In operation, when a surgeon returns to excise previously missed target tissue, the position of the positive margin can be clearly identified and efficiently removed with minimal loss of healthy tissue.

In one aspect, the expandable structure 102 can comprise a balloon. In another aspect, the expandable structure can comprise a polymer. It is further contemplated that the polymer can comprise a thermoplastic elastic polymer and, in an even further aspect, that the thermoplastic elastic polymer comprise polyester polyurethane. In additional or alternative aspects, the polymer can comprise a radio-opaque material and, in further aspects, the radio-opaque material can comprise, for example and without limitation, tantalum, tungsten, rhenium, titanium, oxides of titanium and barium salts.

By way of further explanation, one skilled in the art will recognize that many suitable materials for use in the expandable structure exist. See, for example and without limitation, U.S. Pat. No. 8,287,442 which discloses the use of multiple layers of the inflatable cavity filling member that can be formed of a thermoplastic elastomeric polymer such as polyester polyurethane, e.g., Pellethane™ which is available from Dow Chemical. In one aspect, the polymeric material can have a Shore Durometer of 90 A or less. Other suitable polymeric materials can be employed. The polymeric material of the balloon layers can be a blend of polymers or a copolymer. Balloons of this type are often filled with a radio-opaque fluid for visualization for positional and symmetry verification and CT for positional verification and radiation dose planning. The balloons themselves can be radiopaque by compounding radiopaque agents into the balloon material, coating the inside and/or outside surfaces of a balloon layer with radio-opaque material or providing a radiopaque material between balloon layers. Radiopaque agents or materials may be one or more metals of the group consisting of tantalum, tungsten, rhenium, titanium and alloys thereof or compounds containing oxides of titanium or barium salts such as those which are often used as pigments” U.S. Pat. No. 8,287,442 is herein incorporated by reference in its entirety.

In other aspects, the expanded expandable structure can be left in place after target tissue removal.

In one aspect where the expandable structure is left in place after target tissue removal, the expandable structure can also be designed to facilitate breast tissue regeneration. Here, it is contemplated that the expandable structure can be formed from a matrix that can facilitate tissue regeneration, e.g., breast tissue regeneration. As used herein, the term “matrix” or “scaffold” can be used interchangeably and should be construed to refer a material that is designed to mimic the underlying extracellular matrix of the body most often used to enhance healing or regeneration and “pre-seeded matrix” should be construed to refer a matrix or scaffold that has stem cells or tissue inserted for the purpose of enhancing healing or regeneration. It is contemplated that the outer surface of the expandable structure or the expandable structure itself can be adapted to provide nutrients to a pre-seeded matrix and the matrix can be pre-seeded with, e.g., breast tissue. In a further aspect, the expandable structure can be adapted to prevent or reduce the extent of scar formation components from the wound. In light of the present disclosure, one skilled in the art will appreciate that a suitable matrix seeded with breast tissue can be fabricated by any number of techniques well known in the art, for example and without limitation, the pre-seeded matrix can be printed with cell printing technology as described in U.S. Patent Application Publication No. 2004/0237822 to Thomas Boland, et al., which is hereby incorporated by reference in its entirety. In even further aspects, the expandable structure can comprise a liquid having randomly aligned fibers or a hydrogel or a mesh or a scaffold.

In one aspect where the expandable structure is left in place after target tissue removal, the expandable structure can be a matrix material that gels after insertion in to the breast such as that disclosed in U.S. Patent Publication No. 2012/0130489 to Ary S. Chernomorsky, et al., which is hereby incorporated by reference in its entirety. Alternatively, the expandable structure can comprise a material that, under the influence of an external field (magnetic, ultrasonic, electric, or other) can be caused to align in such a way as to better facilitate regeneration. For one illustrative example, see J Biomed Mater Res A. 2012 September; 100(9):2278-86. doi: 10.1002/jbm.a.34167. Epub 2012 Apr. 12.

In another aspect, the expandable structure 102 can comprise a mesh.

In another aspect, the expandable structure 102 can comprise a biodegradable material. In a further aspect, the biodegradable balloon can be filled with materials that mimic the extracellular matrix of normal tissue in order to allow regeneration of tissue using any number of techniques well known in the art. In one illustrative example, Strattice™ Reconstructive Tissue Matrix, an acellular reconstructive tissue matrix designed to support tissue regeneration, can be used. This acellular reconstructive tissue matrix is derived from porcine dermis, which undergoes non-damaging processing to remove cells and significantly reduce the presence of components believed to play a major role in the xenogeneic rejection response.

In other aspects, the navigation markers 110 on the expandable structure 102 comprise a set of at least one of, for example and without limitation, unique letters, numbers, symbols, designs and the like. In other aspects, the navigation markers can be radio-opaque. In operation, an expandable structure that has navigation markers can enable the precise return to a margin location corresponding to a positive margin located on the excised target tissue.

FIG. 3 shows the excised tissue 300 with the location of a spot found to have remaining cancer or a positive margin 310 by pathological examination. In operation, the excised tissue can be placed in a fixing solution and imaged by any number of commercially available 3-D scanners or laser or optical profilers to develop a three-dimensional model of the excised tissue 330. Next, the 3-D shape of the conformed expandable structure can be measured by any number of imaging techniques including, but not limited to 3-D scanners, cameras, MRI, CAT scan, x-ray and the like. Here, the surface of the expandable structure can be marked with navigational markers in a manner allowing the navigation markers to be seen by x-ray (radio-opaque) or ultra-sound or MRI or other imaging modalities so that once the pathology result is available for the excised target tissue, any area that presents a positive margin can be marked on a computer aided representation of the target tissue by the pathologist or by the digital pathology software and then referenced back to an x-ray of the post-surgical wound cavity using any number of CAD or digital pathology systems. Once the 3-D models are referenced to each other either manually or in software, a surgical marker can then be placed at the exact location to guide removal of remaining cancer. The surgical markers can be placed in the wound cavity adjacent to the mark on the balloon corresponding to the location where cancer was found in pathology to guide the subsequent surgical removal of remaining cancer. In operation, for example, a surgical marker can be placed at the position of the navigation marker identified as the number 2. A subsequent procedure, such as surgery or biopsy or endoscopic procedure can then be used to remove the remaining cancer.

In other aspects and once the target tissue is completely removed, the anatomical referencing system 100 can be adapted for subsequent use to deliver a treatment. In one aspect, the expandable structure 102 and controller assembly 104 can be adapted to deliver a chemotherapeutic agent. Here, the expandable structure 102 can comprise a semi-permeable membrane. In another aspect, the anatomical referencing system can be adapted to further provide radiation treatment. If the target tissue is completely removed, it is contemplated that the expandable structure can be left in place and filled with silicone or some other material configured to mimic the native tissue to form a temporary or permanent implant. In a further aspect, the implant comprises a breast implant.

In other aspects, the present disclosure provides for optimal breast reconstruction. Here, a mastectomy that preserves a breast skin envelope is performed. Next, a prosthesis comprising an expandable structure can be inserted into the breast and can be inflated to preserve the shape of the breast skin envelope. In one exemplary aspect, a needle-lock system, coupled to the port of the expandable structure can be used to inject, for example, fluids into the balloon. If a patient requires post-mastectomy radiation, breast reconstruction may be delayed and the prosthesis can remain in the breast cavity during the radiation treatment. The radiation treatment can comprise external beam radiation. Alternatively, the radiation treatment can comprise brachytherapy techniques for treating the internal breast cavity.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. An anatomical referencing system, comprising:
an expandable structure having a port in communication with an interior cavity and a surface adapted for measuring having a plurality of navigation markers; and
a controller assembly operable to communicate with the expandable structure through the port and having a means for expanding the expandable structure to a selected pressure against the interior cavity and a means for recording the dimensions of the interior cavity of the expandable structure;
wherein the expandable structure is adapted to conform to the geometry of the interior cavity upon expansion.

2. The anatomical referencing system of claim 1, wherein the expandable structure comprises a balloon.

3. The anatomical referencing system of claim 2, wherein the balloon comprises a polymer.

4. The anatomical referencing system of claim 3, wherein the polymer comprises a thermoplastic elastomeric polymer.

5. The anatomical referencing system of claim 4, wherein the thermoplastic elastic polymer comprises polyester polyurethane.

6. The anatomical referencing system of claim 3, wherein the polymer further comprises a radio-opaque material.

7. The anatomical referencing system of claim 6, wherein radio-opaque material comprises at least one of tantalum, tungsten, rhenium, titanium, oxides of titanium and barium salts.

8. The anatomical referencing system of claim 7, wherein the expandable structure comprises a mesh.

9. The anatomical referencing system of claim 1, wherein the selected pressure is from about 0.1 to about 70 mmHg.

10. The anatomical referencing system of claim 9, wherein the selected pressure is from about 2 to about 5 mmHg.

11. The anatomical referencing system of claim 1, wherein the selected pressure is not greater than the interstitial pressure of the target tissue.

12. The anatomical referencing system of claim 1, wherein the means for inflating the expandable structure is selected from the group comprising a pressure pump, a syringe, selectively-controlled temperature changes, a selectively-controlled chemical reaction, and a selectively-controlled mechanical force.

13. The anatomical referencing system of claim 1, wherein means for recording the dimensions of the interior cavity of the expandable structure is selected from the group comprising a three-dimensional scanner, a camera, an ultrasound measuring device, an MRI device, a CAT scan device and an x-ray device.

14. The anatomical referencing system of claim 1, wherein the navigation markers comprise a set of at least one of unique letters, numbers, symbols, and designs.

15. The anatomical referencing system of claim 1, wherein the navigation markers are radio-opaque.

16. The anatomical referencing system of claim 1, wherein the means for recording the dimensions of the interior cavity of the expandable structure is further adapted to record the locations of each of the plurality of navigational markers.

17. The anatomical referencing system of claim 1, wherein the expandable structure comprises biodegradable material.

18. The anatomical referencing system of claim 1, wherein the expandable structure and the controller assembly can be adapted to deliver a chemotherapeutic agent.

19. A method for surgical navigation, comprising the steps of:
removing a target tissue having tissue margins from a patient's body to create a cavity having cavity margins;
measuring the dimensional characteristics of the target tissue;
examining the tissue to locate any positive margins;
using the dimensional characteristics of the target tissue and the locations of any positive margins thereon to construct a three-dimensional map of the excised tissue; and
using the three-dimensional map of the excised tissue to guide a surgery.

20. The method of claim 19, further comprising
providing an anatomical referencing system, comprising:
an expandable structure having a port in communication with an interior cavity and a surface adapted for measuring having a plurality of navigation markers; and
a controller assembly operable to communicate with the expandable structure through the port and having a means for expanding the expandable structure to a selected pressure against the cavity and a means for measuring at least one of the topology and the three-dimensional geometry of the surface of the balloon;
wherein the expandable structure is adapted to conform to the geometry of the cavity in which it is placed upon expansion; and
wherein the means for measuring at least one of the topology or the three-dimensional geometry of the surface of the expandable structure is adapted to record the dimensions of the interior cavity of the expanded structure; and
inserting the expandable structure into the cavity;
closing the cavity;
causing the controller to expand the expandable structure to a selected pressure;
causing the controller to record the dimensions of the interior cavity of the expandable structure and the locations of each of the plurality of navigational markers;
creating a three-dimensional map of the cavity having a plurality of navigational markers;
correlating the three-dimensional map of the target tissue and the three-dimensional map of the cavity to reference positive margins on the excised tissue back to the corresponding cavity margins to create a surgical model; and
using the surgical model to guide a surgery.
